(19) United States
(12) Patent Application Publication
(10) Pub. No.: US 2019/0224229 A1
(43) Pub. Date: Jul. 25, 2019

(54) KEFIRAN FOR USE IN REGENERATIVE MEDICINE AND/OR TISSUE ENGINEERING

(71) Applicant: ASSOCIATION FOR THE ADVANCEMENT OF TISSUE ENGINEERING AND CELL BASED TECHNOLOGIES, Braga (PT)

(72) Inventors: Hajer RADHOUANI, Braga (PT); Cristiana DA MOTA MARTINS GONÇALVES, Braga (PT); Rui Luís GONÇALVES DOS REIS, Porto (PT); Joaquim Miguel ANTUNES DE OLIVEIRA, Braga (PT)

(21) Appl. No.: 16/330,340

(22) PCT Filed: Sep. 5, 2017
(86) PCT No.: PCT/IB2017/055338

§ 371 Date: Mar. 4, 2019

(30) Foreign Application Priority Data
Sep. 5, 2016 (PT) 109606

(51) Int. Cl. A61K 31/715; A61L 27/20; A61K 35/28; A61K 9/00; A61K 31/196; A61K 31/736; A61K 31/728; A61K 31/737; A61K 31/734

(52) U.S. Cl. CPC A61K 31/715; A61L 27/20; A61K 35/28; A61K 9/0019; A61K 31/734; A61K 31/736; A61K 31/728; A61K 31/737; A61K 31/196

(57) ABSTRACT

The present disclosure relates to an exopolysaccharide, in particular to Kefiran and its use in regenerative medicine and/or tissue engineering, compositions, scaffolds and the use of Kefiran in regenerative medicine and/or tissue engineering.

### TECHNICAL FIELD

The present disclosure relates to an exopolysaccharide, in particular to Kefiran and its use in regenerative medicine and/or tissue engineering, compositions, scaffolds and the use of Kefiran in regenerative medicine and/or tissue engineering.

### BACKGROUND ART

Kefiran, an exopolysaccharide produced by lactic acid bacteria, has received a great interest due to its generally recognized as safe status and its potential pharmaceutical and biomedical applications.

Kefir, a traditional cultured-milk beverage, is considered one of the oldest methods of both temporary and long term food preservation. It was originated in the Middle and Far East of Asia, 1000s of years ago. This natural probiotic, with a range of health benefits, has been extremely popular in Eastern Europe, where it is regularly administered as probiotic food to patients in hospitals and recommended for infants. It is beginning to gain a worldwide acceptance and popularity as a healthy probiotic beverage; being commonly home fermented from shared grains, but also recently from a commercial product.

Kefir grains, the starter for obtaining the fermented milk kefir, are elastic, slimy, varying from white to light yellow in colour, and with an irregular and lobed-shaped cauliflower-like structure, of different sizes (1 and 3 cm) in length. These grains are composed of proteins and polysaccharides, containing lactic acid bacteria (LAB), acetic acid bacteria and yeast, held together by a matrix of protein and polysaccharide and yeasts involved in the fermentation. LAB such as Lactobacillus are generally recognised as safe (GRAS), and are known to produce extracellular polysaccharides (EPS), which contribute to the texture of the resulting fermented milk.

The main exopolysaccharide of kefir grains, named Kefiran, is mainly produced by Lactobacillus kefiranofaciens but also by several other unidentified species of Lactobacillus. Kefiran is the clear or pale yellow slimy exopolysaccharide and is a water-soluble polysaccharide containing approximately equal amounts of glucose and galactose residues.

These facts are disclosed in order to illustrate the technical problem addressed by the present disclosure.

### GENERAL DESCRIPTION

Kefir is a remarkable probiotic resource, because it comprises a variety of health claims besides its nutritional status. It is important to highlight that, when compared with other polysaccharides, Kefiran has already several important advantages, as reported, such as antibacterial, antifungal, and antitumor properties, among others. For commercial application, to be a competitive product, Kefiran must be produced at high levels of quality, in a low cost media and the isolation procedure must be easy and with high yields. Taking into account these considerations, Kefiran polymer obtained from kefir grains might be a promising alternative for several biomedical applications.

The Portuguese kefir grains growth rate obtained was about 56% (w/w), at room temperature, and the kefir pH after 24 hrs was about 4.6. Regarding the obtained yield of Kefiran polysaccharide extracted from the kefir grains, it was 4.26% (w/w). The Kefiran structural features were revealed in the ¹H-NMR spectrum. The bands observed in the infrared spectrum showed that the Kefiran is a polysaccharide having β-configuration. Kefiran isolated showed a weight average molecular weight (Mw) of 1,520 kDa and a number average molecular weight (Mn) of 214 kDa.

The zeta potential of Kefiran solution (1% w/v) was performed at 25° C. by laser Doppler micro-electrophoresis in a Zetasizer ZS equipment. Kefiran showed to be a neutral polysaccharide with a zeta potential of 0.728 mV and a conductivity of 0.0274 mS/cm. The determined degradation temperature was 86° C., revealing that Kefiran has a low thermal stability, as determined by differential scanning calorimetry (DSC). Regarding the rheological data obtained, Kefiran showed a pseudoplastic behaviour and an interesting adhesive performance.

These results suggest that Kefiran polymer has attractive and interesting properties for a wide range of biomedical applications, such as tissue engineering, wound healing, bioadhesive material, and cells and drug delivery system.

One aspect of the present subject-matter relates to the use of Kefiran in regenerative medicine and/or tissue engineering. Better results were obtained with Kefiran comprising a molecular weight between 200-2,000 kDa, preferably 1,000-1,720 kDa, more preferably 1,300-1,520 kDa.

In an embodiment for better results, the Kefiran may be use in the treatment or prevention of bone, cartilage, cornea, skin, vascular tissue, peripheral nerve, spinal cord or brain diseases.

In an embodiment for better results, the Kefiran may be use in the treatment or prevention of bone/cartilage diseases or defects.

Another aspect of the present subject-matter relates to a pharmaceutical composition comprising Kefiran in a therapeutically effect amount and a pharmaceutically acceptable excipient to use in the treatment of diseases that comprise the regeneration or treatment of tissues.

In an embodiment for better results, the composition may be use in the prevention or treatment of bone diseases or defects, or cartilage diseases or defects; wound healing.

In an embodiment for better results, the composition may be in bone treatment or cartilage treatment, such musculoskeletal diseases.

In an embodiment for better results, the composition may comprise 0.1-50% (w/V) of Kefiran; preferably 0.5-30% (w/V) of Kefiran; preferably 0.25-10% (w/V) of Kefiran, more preferably 0.5-10% (w/V) of Kefiran, more preferably 1-5% (w/V) of Kefiran.

In an embodiment for better results, the composition may comprise Kefiran with a molecular weight between 200-2,000 kDa, preferably 1,000-1,720 kDa, more preferably 1,300-1,520 kDa.

In an embodiment for better results, the composition may further comprise polysaccharide. Preferably the polysaccharide may be selected from the following list: cellulose; alginate, chondroitin sulphate, chitosan, gellan gum, dextran, collagen, guar gum, carrageenan, heparin, mixtures thereof.

In an embodiment for better results, the composition may comprise a further polysaccharide, a hydrogel, a protein, a therapeutic agent or combinations thereof.

In an embodiment for better results, the polysaccharide/hydrogel is selected from the following list: cellulose; alginate; chitosan, chondroitin sulphate, hyaluronic acid, gellan gum, dextran, collagen, guar gum, carrageenan, heparin, mixtures thereof, preferably, alginate or gellan gum, or mixtures thereof; preferably better results, the polysaccharide/hydrogel is selected from a list consisting of: alginate, chondroitin sulphate, hyaluronic acid and gellan gum, or mixtures thereof.

In an embodiment for better results, the therapeutic agent is selected from a list consisting of: antioxidant, anti-inflammatory, antipyretic, analgesic, anticancer, growth-factor, or mixtures thereof; in particular diclofenac [2-(2,6-dichloroamino)phenyl]acetic acid.

In an embodiment for better results, the composition may further comprise a cell culture media, cell or mixtures thereof; in particular stem cell.

In an embodiment for better results, the composition may be administrated as an injectable form.

In an embodiment for better results, the composition may further comprise a hydrogel or a plurality of hydrogels. Preferably, wherein the hydrogel may be selected from a list consisting of carbopol, matrigel, hyaluronic acid, dextran, alginate, chondroitin sulphate, collagen, gellan gum, or mixtures thereof.

In an embodiment for better results, the composition may further comprise an anti-inflammatory agent, an antiseptic agent, an antipyretic agent, an anaesthetic agent, a therapeutic agent, or mixtures thereof.

Another aspect of the present subject-matter is related to a scaffold for the use in regenerative medicine or tissue comprising use of Kefiran or the composition of the present subject-matter.

Another aspect of the present subject-matter is related to a viscosupplement for the use in regenerative medicine or tissue comprising the use of the compound and/or composition of the present subject-matter.

In an embodiment, the Kefiran average of molecular weight is 1,520 kDa.

In an embodiment, the Kefiran number average molecular weight is 214 kDa.

In an embodiment, the Kefiran polydispersity index is 7.107. The polydispersity was measured by GPC-SEC equipment.

In an embodiment, the Kefiran zeta potential is 0.728 mV in a Zetasizer ZS equipment.

In an embodiment, the Kefiran conductivity is defined 0.0274 mS/cm. The conductivity was measured by Zetasizer ZS equipment.

In an embodiment for better results, the compositions can be combined with other excipients or active substances used in the context of veterinarian and human medicine.

The compositions can be administered by various routes, including topical, enteral and parenteral. Parenteral administration routes include intra-arterial, intra-articular, intracavitary, intradermal, intralympathic, intramuscular, intrasynovial, intravenous, or subcutaneous. Enteral routes include oral and gastro-intestinal. Topical routes include application into the skin and mucous membranes.

In a preferred embodiment, the composition is delivered to a patient by intra-articular injection into a diseased cartilage, which can be repeated according to a clinical prescription regime. In an even more preferred embodiment, the composition is delivered by a single intra-articular injection into a diseased cartilage.

Dosage of the composition can be adapted to the administration route, as well as to the patient profile, including age, gender, condition, disease progression, or any other phenotypic or environmental parameters.

The composition may be in a solid form such as an amorphous, crystalline or semi-crystalline powder, granules, flakes, pills, scaffolds, capsules and suppositories. Such a solid form can be converted into a liquid form by mixing the solid with a physiologically appropriate liquid such as solvents, solutions, suspensions and emulsions.

In another aspect, the present invention provides a method of treating a patient with in regenerative medicine or tissue engineering, the method comprising administering an effective amount of Kefiran/composition described above to the patient.

As described above, the composition may be administered by intra-articular injection, for example, into the patient cartilage.

In a further aspect, the present disclosure provides Kefiran to use in regenerative medicine or tissue engineering, for example, bone/cartilage repair. Moreover, the present invention provides the use of Kefiran in the manufacture of a medicament for regenerative medicine or tissue engineering.

In a particular aspect, the invention provides the composition described above to use in therapy. Further, the present invention provides the composition described above to use in the treatment or repair of bone/cartilage. In addition, the present invention provides the use of the composition described above in the manufacture of a medicament to use in regenerative medicine or tissue engineering.

Throughout the description and claims the word “comprise” and variations of the word, are not intended to exclude other technical features, additives, components, or steps. Additional objectives, advantages and features of the solution will become apparent to those skilled in the art upon examination of the description or may be learned by practice of the solution.

### BRIEF DESCRIPTION OF THE DRAWINGS

The following figures provide preferred embodiments for illustrating the description and should not be seen as limiting the scope of present disclosure.

FIG. 1: ¹H NMR spectrum of Kefiran in D₂O at 60° C.

FIG. 2: Infrared spectrum of Kefiran between 500 cm⁻¹ and 4000 cm⁻¹.

FIG. 3: DSC curve of Kefiran, in particular from 20° C. to 300° C.

FIG. 4: Curves of shear viscosity (A) and shear stress (B) versus shear rate of Kefiran samples at 1% (open symbol) and 10% (filled symbol) of concentration.

FIG. 5: Frequency dependence the viscoelastic moduli, loss modulus G″ (open symbol) and storage modulus G′ (filled symbol), of 1% w/v (A) and 10% w/v (B) Kefiran samples, at 37° C.

FIG. 6: Enzymatic resistance to degradation of Kefiran (black) and HA (white) samples (2%, 1% and 0.5% w/V). * Symbols denote statistically significant differences (p<0.05).

FIG. 7: ¹H-NMR spectrum of Kefiran and Kefiran methacrylated in D₂O at 60° C. i) signal corresponding to H from methylene groups from methacrylate, ii) signal corresponding to H from methyl group from methacrylate.

FIG. 8: Diclofenac release profile from Kefiran hydrogel.

FIG. 9: Injection force of Kefiran formulations and controls.

FIG. 10: Kefiran formulation cryogels. All the cryogels were obtained using silicone molds (h 2 mm×d 8 mm).

FIG. 11: Kefiran formulation scaffolds and controls. All the scaffolds were obtained using a 96-well microplate.

FIG. 12: Kefiran cytotoxicity using a cell line. (a) Cells growth assessed by MTS assay of L929 cells cultured during 72 hrs with Kefiran extracted using different protocols. (b) cells proliferation assessed by dsDNA quantification of L929 cells cultured during 72 hrs with Kefiran extracted using different protocols. Symbols denote statistically significant differences (p<0.05) in comparison to: (§) K2S, (¥) K2L, (*) K3. # denotes statistical significant differences along the time of culture. (c) Cell damage assessed by F-actin staining (cytoskeleton, red) and counterstained with DAPI staining (nuclei, blue), during 72 hrs of culture (scale bar: 50 μm).

FIG. 13: Kefiran cytotoxicity using human primary cells. (a) Cells growth assessed by MTS assay of hASCs cultured during 72 hrs with Kefiran extracted using different protocols. Symbols denote statistically significant differences (p<0.05) in comparison to: (§) K2S, (*) Ctrl−. # denotes statistical significant differences along the time of culture. (b) Cells damage assessed by F-actin staining (cytoskeleton, red) and counterstained with DAPI staining (nuclei, blue), during 72 hrs of culturing (scale bar: 50 μm).

FIG. 14: Kefiran scaffolds biocompatibility. Metabolic activity of human primary cells cultured on scaffolds during 72 hrs and normalize by dsDNA content. Symbols denote statistically significant differences (p<0.05) in comparison to: (¥) 100% Kefiran 1, (#) Kefiran/Alg scaffolds, (§) Ctrl, (*) 72 hrs.

### DETAILED DESCRIPTION

The present disclosure relates to an exopolysaccharide, in particular to Kefiran and its use in regenerative medicine and/or tissue engineering, compositions, scaffolds and the use of Kefiran in regenerative medicine and/or tissue engineering.

In the present disclosure, the physicochemical and structural characterizations of Kefiran exopolysaccharide isolated and purified from Portuguese kefir grains were performed.

Kefiran may be obtained using different methods already disclosed in literature. In the present disclosure, Kefiran was produced and extracted as follows.

In an embodiment, the milk kefir was produced in the following way: kefir grains, in particular 50 g of Kefir grains, used as a starter culture were purchased from a household in Guimarães, Portugal. The grains were cultured, in particular in skimmed milk in a closed-sealed glass container at room temperature overnight, and the medium was exchanged daily for a new culture. This procedure was continued, in particular for 15 subsequent days in order to maintain the grain's viability.

In an embodiment, the milk kefir grain mass was determined as follows: after opening the glass bottles, their culture milk-product kefir grains were sieved, in particular in a plastic filter with 0.25 cm pore size to separate the milk kefir grains from the fermented milk. The milk kefir grains were washed, in particular with 200 mL of sterile saline solution, and the total milk kefir grain mass, in particular wet mass was weighed. After determining the kefir grains mass, a certain amount of grains was used for the Kefiran isolation procedure.

In an embodiment, the acidification kinetics was determined, in particular throughout the fermentation, kefir samples were regularly taken and the pH measured with a digital pH-meter (HANNA, Model HI5222-02). The pH meter was calibrated with standard buffer solutions of pH 4.0 and 7.0 before measuring the fermented kefir samples.

In an embodiment, the isolation of Kefiran polysaccharide was conducted. The Kefiran in the kefir grains was extracted by a method previously described (Piermaria et al. 2009) with some modifications. Briefly, a weighed amount of kefir grains, in particular 20 g was treated in boiling water, in particular in a ratio of (1:10) for 30 min, with discontinuous stirring. The mixture was centrifuged at 18,300 G for 20 min at 20° C. The polysaccharide in the supernatant was precipitated by addition of two volumes of cold ethanol and left at −20° C. overnight. The mixture was once again centrifuged at 18,300 G for 20 min but at 4° C. Pellets were dissolved in hot water and the precipitation procedure repeated twice. The precipitates were re-dissolved in water at 55° C. and the resulting solution concentrated for yielding a crude polysaccharide.

In an embodiment, the Kefiran polysaccharide extracted was frozen at −80° C. or immediately used for dialysis and lyophilisation.

In an embodiment, after dialysis and lyophilisation, Kefiran polysaccharide samples are kept in desiccator to prevent adsorption of moisture.

In an embodiment, the chemical characterization of the isolated exopolysaccharide Kefiran was conducted, as follows: Kefiran was solubilized in particular in 1% (w/V) with deuterium oxide (Sigma Aldrich) at room temperature, and 700 μL of this solution was transferred to a NMR sample tube. ¹H-NMR spectra were recorded on a Varian Unity Plus (Varian, USA) spectrometer; at 60° C. using a resonance frequency of 400 MHz. Chemical shifts are reported in ppm (δ). MestReNova Software 9.0 (Mestre-lab Research) was used for spectral processing.

In an embodiment, Fourier transform infrared spectroscopy (FTIR) was conducted: samples of Kefiran were mixed with potassium bromide and molded into a transparent pellet using a press (Pike, USA). Transmission spectra were acquired on an IR Prestige-21 spectrometer (Shimadzu, Japan), using 50 scans, a resolution of 4 cm⁻¹ and a wavenumber range between 4,400 and 400 cm⁻¹.

The physical characterization of the isolated exopolysaccharide Kefiran was conducted.

In an embodiment, the determination of the molecular weight of the Kefiran samples was carried out by Gel Permeation Chromatography-size exclusion chromatography (GPC-SEC). GPC measurements were performed with a Malvern Viscotek TDA 305 with refractometer, right angle light scattering and viscometer detectors on a set of four columns: pre-column Suprema 5 μm×50 S/N 3111265, Suprema 30 Å 5 μm 8×300 S/N 3112751, Suprema 1000 Å 5 μm 8×300 S/N 3112851 PL and Aquagel-OH MIXED 8 μm 7.5×300 S/N 8M-AOHMIX-46-51, with refractive index detection (RI-Detector 8110, Bischoff). The system was kept at 30° C. The eluent is composed by 0.1 M NaN₃, 0.01 M NaH₂PO₄ (pH 6.6) and used with a flow rate of 1 mL/min. The elution times and the infrared detector signal were calibrated with a commercial calibration polysaccharide set from Varian that contains 10 Pullulan calibrants with narrow polydispersity and Mp (molecular mass at the peak maximum) ranging from 180 Da to 708 kDa.

In an embodiment, DSC experiments were conducted using TA-Q100 equipment, under a nitrogen atmosphere. The samples were prepared and packed in aluminium pans, in particular 5-10 mg of Kefiran polysaccharide. An empty aluminium pan was used as reference. The samples were heated in two stages at a constant heating rate of 20° C./min from 20° C. up to 300° C., then were left at this temperature for a period of 2 min and cooled at 20° C./min to the initial temperature. At this point a second heating run was conducted.

In an embodiment, rheological analyses were performed using a Kinexus pro+ rheometer (Malvern Instruments, UK), using the acquisition software rSpace. The measuring system used in these experiments is composed by the stainless steel cone (40 mm of diameter and 4° of cone angle) and plate geometries. The surface geometry was covered with dodecane to prevent water loss.

In an embodiment, rotational experiments were performed in order to obtain shear viscosity as a function of the shear rate, from 0.01 s⁻¹ to 1,000 s⁻¹, in particular at 37° C. All plots are obtained by the average of at least 3 experiments. These experiments were conducted with Kefiran samples at different concentrations 1% and 10% (w/V) in H₂O.

In an embodiment, the oscillatory experiments were performed to obtain frequency sweep curves. All plots are obtained by the average of at least 3 experiments. These experiments were conducted with Kefiran samples at different concentrations 1% and 10% (w/V) in H₂O.

In an embodiment, pull away experiments were conducted, in particular this experiment involved loading a sample and then pulling away the upper plate at a defined gap speed, in particular at 1 mm/s, with a contact time of 2 s and a contact force of 1 N. The resultant normal force was then recorded as function of gap and was used to determine the adhesion, in particular the area under the force-gap curve.

All quantitative experiments are run in triplicate and results are expressed as a mean ±standard error for n=3. Differences between the groups with p<0.05 were considered to be statistically significant.

In an embodiment, the kefir grains, in particular 10% w/V were cultured in skimmed milk and a growth rate of the kefir grains of about 56% (w/W) was obtained after a 15-day culture period at room temperature.

In an embodiment, the pH of the kefir after 24 hrs was acid, in particular with a pH of 4.62±0.618 at room temperature. The kefir pH reported in the literature is usually reported between 4.2 and 4.6 (Botelho et al. 2014). Other studies reported a lower the pH which reached 3.9 (Pop et al. 2015; Zajsek and Gorsek 2011). This lower value of pH is possibly due to the presence of some components, such as carbon dioxide, acids, lactose, ethanol, proteins and fat contents, among others.

Regarding the obtained yield of Kefiran polysaccharide extracted from the kefir grains, it showed an interesting value of 4.26% (w/w). A slightly lower yield of extracted Kefiran (3.16%, w/w) was observed in other report by Zajsek and Gorsek (Zajsek and Gorsek 2011).

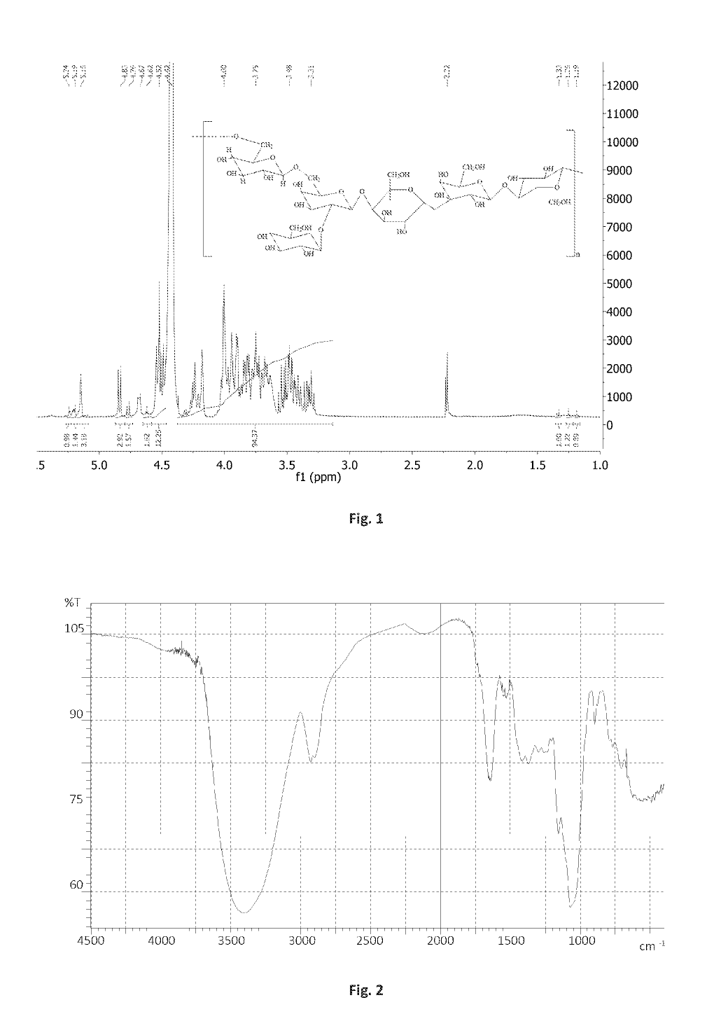

The ¹H nuclear magnetic resonance spectroscopy (¹H-NMR) spectroscopy is a fundamental tool when studying the chemistry of polysaccharides. The use of ¹H-NMR spectroscopy, over other techniques such as chromatography, is characterized by some interesting advantages, i.e. easy sample preparation, easy equipment calibration, fast obtained results, among others. FIG. 1 showed the ¹H-NMR spectrum of the polysaccharide Kefiran in D₂O at 60° C.

The ¹H-NMR spectrum showed a peak at 5.15 ppm for an anomeric β hydrogen, and showed also six signals at the chemical shifts of 4.85 ppm, 4.83 ppm, 4.78 ppm, 4.76 ppm, 4.67 ppm and 4.62 ppm for several anomeric α hydrogens, assigned to a sugar on a lateral branch.

In order to further characterize Kefiran isolated from kefir grains and to identify the fundamental groups present in its structure, IR analysis was performed (FIG. 2).

There were several peaks from 3,400 to 900 cm⁻¹. In the IR spectrum shown in FIG. 2, a band was found at 3,430 cm⁻¹ that corresponds to the hydroxyl groups. In fact, this band region was attributed to the stretching vibration O—H in the constituent sugar residues. The band at 2,930 cm⁻¹, which is associated with the stretching vibration of C—H in the sugar ring, can be assigned to methyl and methylene groups. The band at 1,700 cm⁻¹ was due the stretching vibration of C═O and carboxyl groups. The band around 1,400 cm⁻¹ was attributed to CH₂ and OH groups. The region of 1,100-1,150 cm⁻¹ has showed intense absorptions, a characteristic of stretches C—O—C, and alcohol groups in carbohydrates. The presence of band at 900 cm⁻¹ indicated β-configuration. According to several authors, these several bands indicate that the compound is a polysaccharide (Botelho et al. 2014; Ghasemlou et al. 2011).

The molecular weight of the Kefiran extracts was determined by size exclusion chromatography (GPC-SEC). The molecular weight of a substance, particularly a polymer, is a key chemical characteristic that can dramatically influence the material mechanical performance, particularly the viscosity and rheological behaviour. In this sense, size exclusion chromatography was used to determine the number-average molecular weight (Mn) and weight-average molecular weight (Mw) for Kefiran polysaccharide.

In an embodiment, Kefiran polysaccharide showed an average of molecular weight of 1,520 kDa and a number average molecular weight of 214 kDa, with a polydispersity index of 7.107. It is important to highlight that the polydispersity index of natural polysaccharides is usually in the range of 1.5˜2.0. Using fraction techniques, i.e. size exclusion chromatography and fractional precipitation or even ultrafiltration using different pore size membranes, is possible to obtain a lower polydispersity index (close to 1).

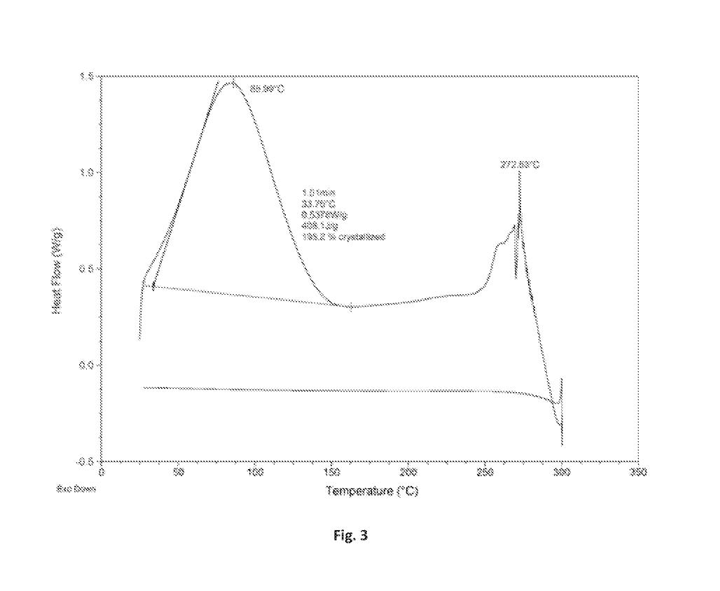

DSC is a thermal analysis technique, relevant in the characterization of the thermal properties and transitions of a polymer. DSC was performed to Kefiran samples with two straight runs (FIG. 3).

In an embodiment, in particular in FIG. 3 it is presented the Kefiran thermogram (endothermic heat flow), which exhibited sharp endothermic peak at 86° C. This transition (86° C.) could be related with loss of H₂O, being explained by the hydrophilic nature of Kefiran functional groups; the presence of this peak can also reveal the existence of water bound and also the destruction of the Kefiran sample that did not recover, as observed in the second run. Kefiran herein showed lower thermal stability as compared to other that reported a degradation temperature of 352° C. (Botelho et al. 2014).

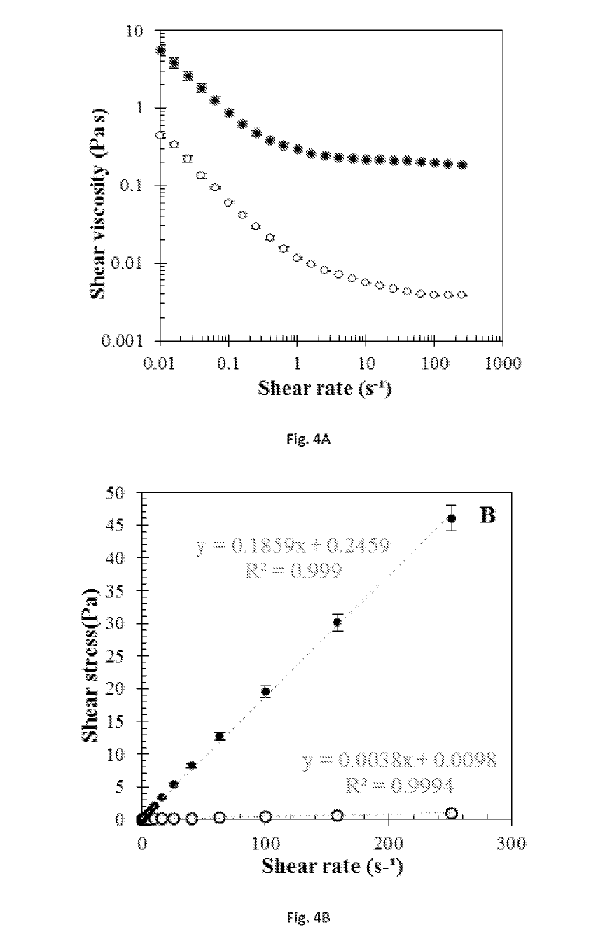

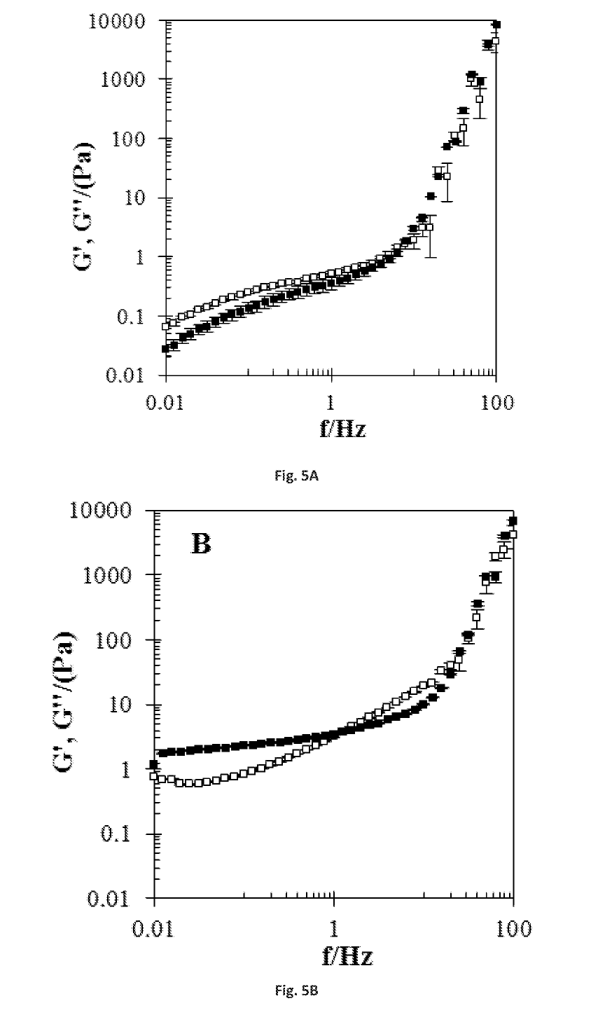

Flow behaviour of Kefiran solutions, in particular 1% (w/V) and 10% (w/V) Kefiran solutions, is illustrated in FIGS. 4A and 4B. FIG. 4A presented the shear viscosity function to the shear rate.

Shear viscosity of Kefiran samples at different concentrations decreased with the increment of shear rate. It showed that Kefiran samples (1% and 10%, w/v) presented shear-thinning (or pseudoplastic) behaviour at 37° C.

FIG. 4B presented the shear stress function to the shear rate. It demonstrated the relation between the shear stress applied and the resulting shear rate. Kefiran samples in particular 1% (w/V) and 10% (w/V) revealed an infinite viscosity until a sufficiently high stress as applied to initiate flow. Above this stress the biomaterial then indicated simple Newtonian flow, which was identified as a Bingham plastic model.

The mechanical spectra of 1% (w/V) and 10% (w/V) of Kefiran were obtained (FIG. 5, A and B, respectively), at 37° C.

Storage and loss moduli are both functions of frequency and give an insight of the structure of polymer solutions. The Kefiran solution at 10%, w/V started by an elastic behaviour. More particularly, Kefiran sample presented a cross over at 1.6 Hz, changing from a phase angle of about 25.7° (elastic/gel) to an average of approximately 58° (viscous liquid). In this case, the mechanical spectra demonstrated the behaviour of a polysaccharide solution at low frequency where the loss modulus is greater than the storage modulus. In fact, both moduli increased with frequency swept but G″ increased more rapidly and tends to overcome G′ at higher frequencies. This statement could be explained by the mechanism of an entrapped network system at low frequencies. At high frequencies, the polymer chains do not have sufficient time to uncoil and slide and therefore may behave like a gel (G′>G″).

In an embodiment, preliminary pull away tests to 1% (w/V) and 10% (w/V) Kefiran samples show that Kefiran polysaccharide is characterized by an adhesive performance. Being, this effect 97% higher for 10% solutions, with an adhesion of 1.159±0.018 N/s. Kefiran solutions of 1% (w/V) presented a similar adhesion value to water, 0.135±0.049 N/s. In fact, polysaccharides such as Kefiran may help bacterial cells for adhesion to biological surfaces and biofilm formation. It is important to point out that the advances in biofilm formation knowledge, coupled with emerging engineered biomaterials, provide many potential platforms and strategies to prevent or significantly reduce biofilm infections.

Therefore, Kefiran is an important material to use in medicine, especially for tissue engineering by replicating the mechanical and viscoelastic characteristics of tissues such as cartilage, cornea, skin, vascular tissue, peripheral nerve, spinal cord, brain, among others.

Kefiran polysaccharide of the present disclosure, with both viscous and elastic properties, surprisingly may be used as a scaffold or as a viscosupplement product with several interesting properties. In fact, this novel injectable biomaterial can be used alone or in combination with cells, for intra-articular pathologies.

In fact, Kefiran polysaccharide of the present disclosure surprisingly with its several potential properties such as high molecular weight, viscoelastic characteristics, high biocompatibility, biodegradability and bioadhesiveness, is modulated to achieve, for example, an adequate structure as demanded by articular cartilage defects applications. The articular cartilage disease such as osteoarthritis (OA) is not confined to any particular geographical area and is affecting the entire humankind. The direct and indirect economic costs of OA are excessive and in this sense, there is an urgent need to develop a new efficient and economically competitive therapy to manage and treat OA.

Kefiran polysaccharide of the present disclçosure with its cells/nanoparticles/genes/drugs encapsulating ability and bioadhesiveness properties could be modulated to achieve, for example, an adequate adhesiveness to several tissues in order to allow the appropriate cells/nanoparticles/genes/drugs delivery.

In an embodiment, the reducing power activity of Kefiran (1% w/V) is 8.47 μg ascorbic acid equivalent per mL of sample solution. Hyaluronic Acid (HA) does not present any reducing power activity.

In an embodiment, the iron chelating activities of Kefiran and HA are 26.66% and 28.5%, respectively at a concentration of 1% w/V.

In an embodiment, the hydroxyl radical scavenging activities of Kefiran and HA are 73.66% w/V and 74.44% w/V, respectively at a concentration of 1% w/V.

In an embodiment, the superoxide radical scavenging activities of Kefiran and HA are 25.99% w/V and 19.05% w/V, respectively at a concentration of 1% w/V.

In an embodiment, the nitric oxide radical scavenging activity of Kefiran is 40.91% at a concentration of 1% w/V. HA does not present any nitric oxide scavenging activity.

In an embodiment, Kefiran does not present antimicrobial activity for Staphylococcus aureus ATCC 6538, Staphylococcus epidermidis ATCC 14990 and Pseudomonas aeruginosa ATCC 27853 after 24 hrs of inoculation.

In an embodiment, Kefiran presents a resistance to hyaluronidase.

In an embodiment, extrusion force of hyaluronic acid, measured by an injectability measurement device at room temperature (20° C.), is 11.3 N, which is the highest one in all the Kefiran formulations.

In an embodiment, the extrusion force, measured by an injectability measurement device at room temperature, of Kefiran is 1 N.

In an embodiment, the smoothest injection is the formulation 75% Kefiran+25% Chondroitin Sulphate which has an extrusion force of 0.09 N.

In an embodiment, the cryogels obtained are those composed by Kefiran+Gellan Gum.

In an embodiment, the Kefiran polysaccharides K1 (from extraction method 1) and K2 (from extraction method 2) present higher number of living L929 cells after 48 hrs and 72 hrs.

In an embodiment, the Kefiran polysaccharides K3 (from extraction method 3) present the lower number of living L929 cells after 48 hrs and 72 hrs.

In an embodiment, Kefiran 1—Kefiran extracted from method 1, was the exopolysaccharide that shows the best properties comparing to Kefiran 2—Kefiran extracted from method 2 and Kefiran 3—Kefiran extracted from method 3.

In an embodiment, after 24 hrs of incubation, a higher metabolic activity of hASCs cells with Kefiran 1 (75% w/V or 50% w/V) +Gellan Gum (25% w/V or 50% w/V) than Kefiran 1 (75% w/V or 50% w/V)+Alginate (25% w/V or 50% w/V) scaffolds was observed.

In an embodiment, after 72 hrs of incubation, a higher metabolic activity of hASCs cells with Kefiran 1 (75% w/V or 50% w/V)+Gellan Gum (25% w/V or 50% w/V) than Kefiran 1 (75% w/V or 50% w/V)+Alginate (25% w/V or 50% w/V) scaffolds was observed.

In an embodiment, after 24 hrs and 72 hrs of incubation, the scaffold that shows the higher metabolic activity of hASCs cells was Kefiran 1 (75% w/V)+Gellan Gum (25% w/V) than 100% w/V Kefiran 1 scaffolds.

Due to its aforementioned remarkable properties, Kefiran is surprisingly a candidate for the preparation of such biomaterials. Kefiran biopolymer of the present disclosure allows cell attachment and proliferation, demonstrating that it could substitute for missing or damaged tissue.

### EXAMPLES

The following examples are provided with complete disclosure and description of how to make, use and characterize the best Kefiran formulation hydrogel or scaffold. During the research, three main products were studies: the Kefiran polysaccharides extract, the Kefiran hydrogels with or without other biomaterials and finally the Kefiran scaffolds with or without other biomaterials. Furthermore, three different Kefiran extraction methods were performed in order to guarantee the best Kefiran extract for cell viability.

In an embodiment, for the Kefiran polysaccharide extracts, the antioxidant, anti-inflammatory and antimicrobial activities were studied. Secondly, several formulations of Kefiran hydrogels with other biomaterials were produced; and the injectability assay was performed in order to find the best formulation that could be used as viscosupplement. Thirdly, Kefiran formulations scaffolds were produced, and the cytotoxicity and biocompatibility with different cells assays were performed.

In an embodiment, Kefiran polysaccharides (K1) in the kefir grains were isolated by a method previously described (Piermaria, Pinotti et al. 2009) with major modifications. Briefly, kefir grains (20 g) were treated in boiling water (1:10) for 30 min, with discontinuous stirring. The mixture was centrifuged at 18,300 G for 20 min at 20° C. The polysaccharides in the supernatant were precipitated by addition of two volumes of cold ethanol absolute and left at −20° C. overnight. The mixture was again centrifuged at 18,300 G for 20 min at 4° C. Pellets were dissolved in hot water during 5 hrs and the precipitation procedure repeated three times. The precipitates were dissolved in water at 60° C. and the resulting solution concentrated for a crude polysaccharide. Kefiran polysaccharides extracted were frozen at −80° C. After dialysis and lyophilisation, Kefiran polysaccharides were kept in desiccator to prevent adsorption of moisture.

In an embodiment, it was shown the antioxidant activity of Kefiran polysaccharide (as compared to a gold standard biomaterial—Hyaluronic Acid) which is highly relevant as it may induce cell protection in contexts of high oxidative stress.

In an embodiment, Kefiran and Hyaluronic Acid (HA) samples were prepared in different concentrations (1% and 0.5%, w/V). The samples were diluted in H₂O; and several assays were performed to determine their antioxidant properties.

In an embodiment, for reducing power assay—(RPA)—(Qi, Zhang et al. 2006, Singhal and Ratra 2013), the Kefiran samples were incubated with potassium ferricyanide (2% w/V) at 50° C. for 20 min. Reaction was terminated by the addition of trichloroacetic acid (10% w/V) and centrifuged at 4,700 G (10 min). The obtained supernatant was mixed with H₂O₂ and ferric chloride (1% w/V), and the absorbance was acquired at 700 nm. The test was carried out in triplicate and ascorbic acid was used as standard for comparison.

In an embodiment, the reducing power activity of the Kefiran and HA samples was expressed of the Ascorbic Acid Equivalent Reducing Capacity (AAEC).

Reducing power activity assay consists of measuring the electron-donating capacity of an antioxidant compound using the potassium ferricyanide reduction approach. Presence of reducers causes the conversion of the Fe³⁺/Ferricyanide complex to the ferrous from which acts as an important indicator of its antioxidant performance (Aparadh, Naik et al. 2012). Herein, the reducing power assay of Kefiran and HA has been performed and the results were shown in the Table I.

TABLE I
Reducing power activity of Kefiran and HA samples

| | Ascorbic Acid Equivalent Capacity (AAEC) |
|---|---|
| Samples (% w/V) | (μg/mL of sample solution) |
| Kefiran 1% | 8.47 ± 0.04 |
| Kefiran 0.5% | 4.44 ± 0.05 |
| HA 1% | — |
| HA 0.5% | — |

In an embodiment, Kefiran of the present disclosure showed an interesting reducing power activity. Contrary to Kefiran, hyaluronic acid did not show any reducing power activity.

In an embodiment, was evaluated the metal chelating activity (MCA) of Kefiran. For ferrous ion chelating ability (El and Karakaya 2004) of Kefiran and Hyaluronic Acid samples, a reaction mixture, containing 154 μL of Kefiran samples (1% w/V), iron(II) chloride (15 μL, 2 mM), was shaken well and incubated for 60 min at room temperature (RT). Ferrozine (31 μL, 5 mM) was added and the absorbance of the mixture was measured at 562 nm against the blank. The test was carried out in triplicate and EDTA (Ethylenediaminetetraacetic acid) was used as standard.

The percentage of inhibition of ferrozine-Fe²⁺ complex formation was given in the following formula:

Ferrous ions chelating ability (%)=[1−(ABSₛₐₘₚₗₑ/ABS_{control})]×100

ABSₛₐₘₚₗₑ Absorbance in the presence of the sample

ABS_{control} Absorbance of the control solution (containing all reagents except Kefiran or HA)

One of the mechanisms of antioxidant defence is chelation of transition metals, thus preventing catalysis of Fenton reactions and hydroperoxide decomposition. The principal strategy to avoid ROS generation that is related with redox active metal catalysis implicates chelating of the metal ions (Flora 2009, Aparadh, Naik et al. 2012).

In an embodiment, the results of the ferrous ion-chelating effect of Kefiran and HA were shown in Table II. The ferrous ion-chelating effect of Kefiran and HA samples were low, compared to EDTA.

TABLE II
Metal Chelating Activity of Kefiran and HA samples

| | Iron Chelating | |
|---|---|---|
| Samples (% w/V) | Capacity (%) | mg EDTA per gram of sample |
| Kefiran 1% | 26.66 ± 0.05 | — |
| Kefiran 0.5% | 20.19 ± 0.02 | — |
| HA 1% | 28.5 ± 0.04 | — |
| HA 0.5% | 24.05 ± 0.05 | — |

In an embodiment, the Kefiran showed 27% Fe²⁺ ion chelating ability at 10 mg/mL where the standard EDTA showed 91.1% at a lower concentration (1.5 mg/mL). It is important to highlight that Kefiran polysaccharide presented a same behaviour as ascorbic acid which has limited iron chelation potential itself.

In an embodiment, scavenging activity of hydroxyl radical—Hydroxyl Radical Scavenging Activity (HRSA)—of Kefiran and Hyaluronic Acid samples was assayed by deoxyribose method (Nagai, Nagashima et al. 2005) with major modifications. Reaction mixture contained 0.45 mL of sodium phosphate buffer (PBS) (0.2 M, pH 7.0), 0.15 mL of 2-deoxyribose solution (10 mM), 0.15 mL of FeSO₄-EDTA solution (10 mM FeSO₄, 10 mM EDTA), 0.15 mL of H₂O₂ solution (10 mM)); and 100 μL samples were added to the mixture. Solutions were completed to a final volume (1.5 mL) with H₂O then incubated at 37° C. for 4 hrs. Reaction was stopped by adding 0.75 mL of TCA solution (2.8% w/V) and 0.75 mL of TBA solution (1% w/V in 50 mM NaOH solution). Solutions were boiled for 10 min and then cooled. Absorbance was measured at 520 nm against the blank. The test was carried out in triplicate and results were expressed in terms of ascorbic acid equivalent antioxidant capacity (AAEC).

Hydroxyl radical scavenging activity (HRSA) of the extract was also reported as % inhibition of deoxyribose degradation is calculated by using the following formula:

HRSA (%)=[1−(ABSₛₐₘₚₗₑ/ABS_{control})]×100

ABSₛₐₘₚₗₑ Absorbance in the presence of the sample,

ABS_{control} Absorbance of the control solution (containing all reagents except Kefiran or HA).

The hydroxyl radical is the most reactive of the oxygen species and causes significant damage to adjacent biomolecules (Liu, Sun et al. 2015). Various polysaccharides might release hydrogen proton to react with hydroxyl radicals, causing decreased of the rate of hydroxyl radical attack on deoxyribose (Wang, Yang et al. 2012). The results of hydroxyl radical scavenging activity of Kefiran and HA are represented in the Table III.

Kefiran and HA samples showed almost the same capacity to scavenge hydroxyl radicals (73.66% and 74.44%, respectively) at the same concentration.

TABLE III
Hydroxyl Radical Scavenging activity of Kefiran and HA samples

| | | Ascorbic Acid |
|---|---|---|
| | Inhibition percentage | Equivalent Capacity |
| | of hydroxyl | (AAEC) (μg/mL of |
| Samples (% w/V) | radical production (%) | sample solution) |
| Kefiran 1% | 73.66 ± 0.02 | 318.75 ± 0.21 |
| Kefiran 0.5% | 69.29 ± 0.05 | 213.89 ± 0.15 |
| HA 1% | 74.44 ± 0.03 | 337.45 ± 0.34 |
| HA 0.5% | 70.20 ± 0.05 | 235.84 ± 0.14 |

Kefiran, an exopolysaccharide, had hydroxyl radical scavenging activity due to its affinity for the OH radical and did not show significant ferric chelating activity. The same statement was also observed in other sulphated polysaccharide extracted from Pleurotus sajor-caju (Telles, Sabry et al. 2011).

In an embodiment, superoxide scavenging of Kefiran (SRSA) and HA samples was determined by the nitroblue tetrazolium reduction method (Gutierrez and Baez 2014) with major modifications. Reaction mixture consisted of 500 μL of NBT solution, 500 μL NADH solution and 250 μL of samples was mixed. Reaction was started by adding 250 μL of PMS solution to the mixture. After 5 min, absorbance was measured at 560 nm. The test was carried out in triplicate and results were expressed in terms of ascorbic acid equivalent antioxidant capacity (AAEC).

Superoxide radical scavenging activity (SRSA) of the extract was also reported as % inhibition of superoxide radical is calculated by using the following formula:

SRSA (%)=[1−(ABSₛₐₘₚₗₑ/ABS_{control})]×100

ABSₛₐₘₚₗₑ Absorbance in the presence of the sample

ABS_{control} Absorbance of the control solution (containing all reagents except Kefiran or HA)

It was shown that Kefiran presented a higher capacity to scavenge superoxide radicals (23%) than HA at the same concentration (1% w/V) (Table IV).

TABLE IV
Superoxide Radical Scavenging activity
of Kefiran and HA samples

| | Inhibition percentage | |
|---|---|---|
| | of superoxide | AAE (μg/mL of |
| Samples (% w/V) | radical production (%) | sample solution) |
| Kefiran 1% | 25.99 ± 0.03 | 179.59 ± 0.04 |
| Kefiran 0.5% | 13.84 ± 0.02 | 49.52 ± 0.03 |
| HA 1% | 19.05 ± 0.02 | 86.01 ± 0.02 |
| HA 0.5% | 10.71 ± 0.02 | 35.55 ± 0.02 |

The results showed that the Kefiran extract possessed the strongest reducing power and superoxide radical scavenging over HA. In this research, it has been showed that Kefiran have a high antioxidant potential. In fact, this exopolysaccharide showed a distinct antioxidant activity in the majority of in vitro working mechanisms of antioxidant activity comparing to hyaluronic acid.

In an embodiment, anti-inflammatory activity of Kefiran polysaccharides was determined. Nitric oxide (NO) is a significant mediator of various physiologic and pathologic processes. NO, a water- and lipid-soluble gas, is perfectly appropriate as a powerful inflammatory mediator due to its strong reactivity with oxygen, superoxide, and iron-containing compounds (Amin and Islam 2014). Kefiran and Hyaluronic Acid samples were prepared in different concentrations (1% and 0.5% w/V). The samples were diluted in H₂O.

In an embodiment, ability of Kefiran and HA samples to scavenge nitric oxide radical generated in a cell-free system was evaluated in this research. Briefly, 1.5 mL of sodium nitroprusside (10 mM) and 1.5 mL of samples were mixed. The samples were incubated at 37° C. for 150 min. The same volume (3 mL) of freshly prepared Greiss reagent were added to the previous mixture. All measurements were taken in triplicate. Absorbance of the chromaphore formed during the diazotization of nitrite with sulphanilamide and subsequent coupling with napthylethylenediamme was read at 546 nm against the blank.

The test was carried out in triplicate and results were expressed in terms of quercetin equivalent antioxidant capacity (QAEC). The amount of nitric oxide radical inhibition (NORS) was also calculated following this equation:

NORS (%)=[1−(ABSₛₐₘₚₗₑ/ABS_{control})]×100

ABSₛₐₘₚₗₑ Absorbance in the presence of the sample

ABS_{control} Absorbance of the control solution (containing all reagents except Kefiran or HA)

The tests show that Kefiran presented an interesting capacity to scavenge nitric oxide radical (almost 41%) comparing to the HA (Table V) that did not present any potency in this assay. The nitric oxide scavenging activity of the Kefiran was 41% at 10 mg/mL, whereas the standard quercetin showed a more pronounced capacity (78.22%) at 1 mg/mL.

TABLE V
Nitric oxide radical scavenging capacity
of Kefiran and HA samples

| | Inhibition percentage | |
|---|---|---|
| | of nitric oxide | QAEC (mg/mL of |
| Samples (% w/V) | radical production (%) | sample solution) |
| Kefiran 1% | 40.91 ± 0.04 | 0.16 ± 0.02 |
| Kefiran 0.5% | 3.92 ± 0.03 | — |
| HA 1% | — | — |
| HA 0.5% | — | — |

In an embodiment, Kefiran polysaccharide exhibited excellent NO scavenging activity leading to the reduction of the nitrite concentration in the assay medium. The results justify a potential advantage of Kefiran over HA as a bioactive molecule for cell protection in oxidative stress environments, including inflammation contexts.

These results demonstrated that Kefiran represented a great scavenger for reactive oxygen species (ROS) and showed also an anti-inflammatory property, which is greatly relevant taking into account its potential pharmaceutical application.

It is important to highlight that the potential contribution of any biomaterial to oxidative stress is very high when assessing biocompatibility of new biomaterials candidates. Oxidative stress is a promoter of cell death and causes severe interferences in the normal physiological functioning of the host. The demonstration of antioxidant and anti-inflammatory properties is important to prove that the biomaterial candidate does not contribute to any pro-oxidative stress. Furthermore, the demonstration of the antioxidant activity of Kefiran (as compared to a gold standard biomaterial—HA) is highly relevant as it may induce cell protection in contexts of high oxidative stress.

In an embodiment, the antimicrobial activity of Kefiran polysaccharides was measured, despite the important improvements made in aseptic surgical procedures, infection due to bacterial contamination remains a challenge for in vivo application of biomaterials. In this research, antimicrobial activities of Kefiran polysaccharides were evaluated for three reference strains Staphylococcus aureus ATCC 6538, Staphylococcus epidermidis ATCC 14990 and Pseudomonas aeruginosa ATCC 27853, S. aureus, S. epidermidis and P. aeruginosa, account together for the majority infection isolates. They represent, in absolute, the main causative agents in orthopedics (Ribeiro, Monteiro et al. 2012).

Bacterial suspensions were plated (overnight) onto Tryptone Soy Agar medium. Twenty μl of each of Kefiran samples (2%, 1%, 0.5% and 0.25% w/V) were tested; and sterile water was used as control for the experiment; the assay was performed in triplicate. The prepared plates were incubated at 37° C. during 18 hrs to 24 hrs. The evaluation of the antimicrobial activity is based on the absence or presence of bacterial growth in the contact zone between culture medium and Kefiran samples; and on the extent of the eventual appearance of a zone of inhibition around the samples. Inhibition zones are measured in millimetres (mm).
Concentration of bacterial suspensions:
   Pseudomonas aeruginosa—6.8×108 ufc/mL;
   Staphylococcus aureus—3.3×108 ufc/mL;
   Staphylococcus epidermidis—2.7×108 ufc/mL.

In an embodiment, the Kefiran samples (2%, 1%, 0.5% and 0.25% w/V) did not present antimicrobial activity after 24 hrs of inoculation.

In an embodiment, it was measured the enzymatic resistance of Kefiran polysaccharides. The hyaluronic acid is the responsible for the high viscosity and thixotropic behavior of synovial fluid. The half-life of the HA formulations is known to be short (<9 hrs) requiring several dosages. HA modifications are being reported with higher half-life, but it is not sufficient and the HA production cost is still too high. It is important to find alternative biomaterial that promise to resist to enzymatic degradation (Saturnino, Sinicropi et al. 2014).

In an embodiment, Kefiran and Hyaluronic acid were subjected to enzymatic reactions with hyaluronidase (EC 3.2.1.35; HAse), under pH 7 in a water bath at 37° C. The samples were cooled in an ice bath in order to stop the enzymatic reaction. The result was measured as the release of reducing sugars, using the DNS method, since studied polysaccharides had reducing ends on the repeating units of the structure. Glucose was used as standard. GPC-SEC was also obtained for some experiments to confirm the reliable use of DNS method to monitor the enzyme action over the polysaccharides. HA was used as the negative control and non-substrate as positive control.

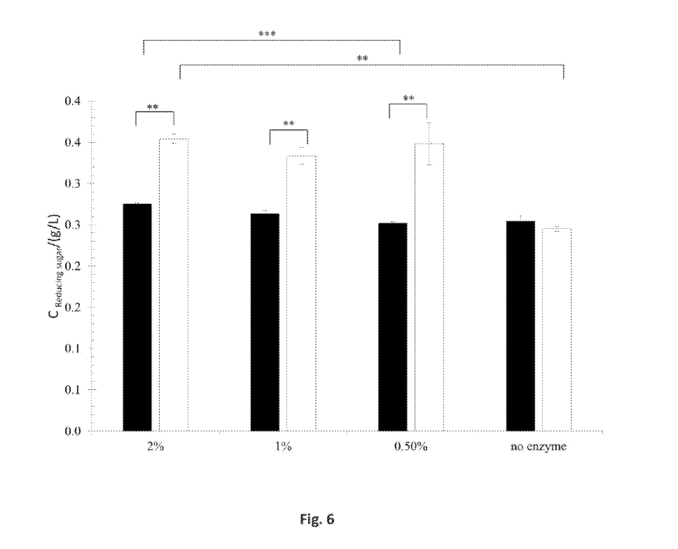

In an embodiment, the results achieved, in terms of reducing sugars release during the enzymatic reactions, for 48 hrs of reaction are presented in FIG. 6. Kefiran exhibited a higher resistance to hyaluronidase degradation than HA that presented a degradation of 30%. GPC-SEC analysis confirmed the results obtained.

In an embodiment, osteoarthritic joint is characterized for expression of enzymes that degrade the cartilage matrix components, as hyaluronic acid. Marketed viscosupplements based on HA are susceptible to Hyaluronidases (HAases) degradation action, resulting in a fast clearance. The evaluation of Kefiran resistance to HAase activity is an important step to confirm advantage over HA.

In an embodiment, it is important to highlight that Kefiran biopolymer could be a promising alternative viscosupplement to HA as it resists to enzymatic degradation.

In an embodiment, the functionalization of Kefiran polysaccharide was performed. Methacrylated Kefiran was synthesized by reacting the Kefiran polysaccharide with methacrylic anhydride (MA). Briefly, 0.2 g of Kefiran was dissolved into 20 mL of PBS (5 mM, pH 7.4). Then, 0.392 mL of methacrylic anhydride was added to this solution at room temperature. The reaction was continued under constant stirring for 2 hrs 30 min. During the reaction, the pH 8.5 was continuing reajusted with NaOH solution (1M).

In an embodiment, the modified Kefiran methacrylated (KefMA) solution was precipitated with 3 times in volume of cold acetone and purified by dialysis for 1 week against distilled water to remove the excess of MA and acetone. Water was completely removed and exchanged at least 3 times per day. All the batches were lyophilized and stored in a dry.

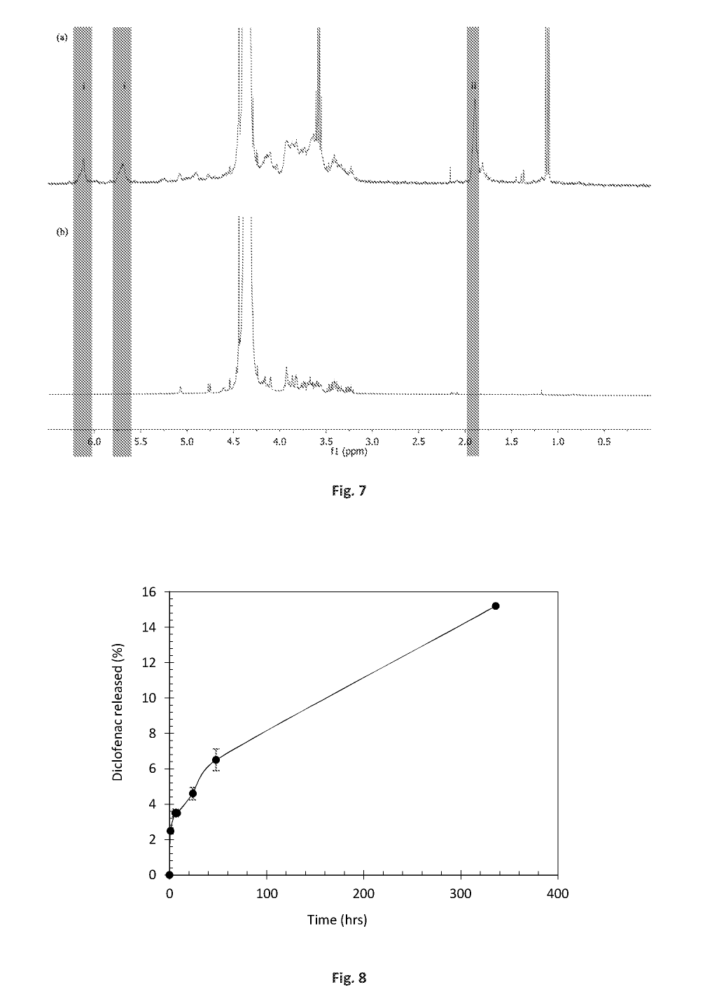

In an embodiment, the chemical modification to Kefiran was assessed by ¹H-NMR spectra (FIG. 7). The methacrylation degree (DM, fraction of modified hydroxyl groups per repeating unit) was determined by the relative integration of the methylene proton peak (Imethylene) of methacrylated groups to methyl protons of the initial standard (ICH₃). The n_{methylene} and n_{CH3} standard correspond to the number of protons in the CH₂=from the methacrylic group and in methyl groups of Kefiran, respectively. The n_{OH} monomer corresponds to the number of reactive —OH sites per sugar residue in the Kefiran structure.

In an embodiment, the degree of methacrylation, which is the fraction of methacrylated hydroxyl groups per average monomer of Kefiran, obtained for the synthesized material was 47.6%±3.8.

In an embodiment, drug delivery of Kefiran-based hydrogels was performed. Diclofenac [2-(2,6-dichloroamino)phenyl]acetic acid is a non-steroidal anti-inflammatory drug. It is used for the treatment of rheumatoid arthritis, osteoarthritis, among others. The ability of Kefiran as polysaccharide-based hydrogels to absorb large amounts of water makes it proper to be used as injectable drug delivery system for localized therapy.

In an embodiment, the release experiments from the Kefiran cryogels were performed in this research. For this purpose, we prepared Kefiran solution (4% w/V) and diclofenac sodium salt solution (0.5 mg/mL) in H₂O. The mix solution (Kefiran+Diclofenac) was transferred to silicone molds h 2 mm×d 8 mm; then the molds were placed immediately in a freezer at −20° C. for 24 hrs and transferred to a refrigerator at 4° C. for a further 24 hrs.

In an embodiment, the release profiles are reported as (Mt/M∞)×100 where Mt represents the amount of model (diclofenac) drug found in the release medium (PBS, pH 7.4) at different time t (T1 hr, T6 hrs, T8 hrs, T24 hrs, T2 Days and T14 Days) and M∞ represents the amount of model drug released after an infinite time, i.e., the total amount of the diclofenac initially (0.5 mg/mL) present in the hydrogel.

In an embodiment, the calibration curve for diclofenac was prepared by taking the absorbance at λ 300 nm of diclofenac standard solutions at different concentrations (0.5 mg/mL-0.01 mg/mL of diclofenac salt sodium). All the studies were carried out in triplicate.

It is important to observe that diclofenac was released slowly (FIG. 8). After 14 days, just 15.2%±0.6% of the diclofenac loaded into Kefiran hydrogels was released. Immediate-release products generally result in relatively rapid drug absorption and onset of accompanying pharmacodynamic effects. In the case of Kefiran-based hydrogels containing diclofenac, the pharmacodynamic activity showed to be slow which could be a potential drug delivery/release system that tend to control better the disease at a minimum dose.

In an embodiment, several formulations of Kefiran hydrogels with other biomaterials (Gellan Gum (GG), Hyaluronic Acid (HA), Chondroitin Sulphate (CS) and Alginate (Alg)) at different concentrations were produced (Table VI). All the biomaterials were prepared at 2% w/V concentration in ultrapure H₂O.

TABLE VI
Different formulations of Kefiran with other biomaterials

| Formulation | Kefiran (2% w/V) | Other biomaterials (2% w/V) |
|---|---|---|
| Formulation 1 (F1) | 75% v/V | 25% v/V of Gellan Gum |
| Formulation 2 (F2) | 75% v/V | 25% v/V of Hyaluronic Acid |
| Formulation 3 (F3) | 75% v/V | 25% v/V of Chondroitin |
| | | Sulphate |
| Formulation 4 (F4) | 75% v/V | 25% v/V of Alginate |
| Formulation 5 (F5) | 50% v/V | 50% v/V of Gellan Gum |
| Formulation 6 (F6) | 50% v/V | 50% v/V of Hyaluronic Acid |
| Formulation 7 (F7) | 50% v/V | 50% v/V of Chondroitin |
| | | Sulphate |
| Formulation 8 (F8) | 50% v/V | 50% v/V of Alginate |
| Formulation 9 (F9) | 25% v/V | 75% v/V of Gellan Gum |
| Formulation 10 (F10) | 25% v/V | 75% v/V of Hyaluronic Acid |
| Formulation 11 (F11) | 25% v/V | 75% v/V of Chondroitin |
| | | Sulphate |
| Formulation 12 (F12) | 25% v/V | 75% v/V of Alginate |

| | |
|---|---|
| Control 1 (C1) | Kefiran 100% |
| Control 2 (C2) | Gellan Gum 100% |
| Control 3 (C3) | Hyaluronic Acid 100% |
| Control 4 (C4) | Chondroitin Sulphate 100% |
| Control 5 (C5) | Alginate |

Injectability is a key-product performance parameter of any parenteral dosage form. This assay included pressure or force required for injection, evenness of flow, and freedom from clogging such as no blockage of the syringe needle (Cilurzo, Selmin et al. 2011).

In an embodiment, the injectability assay of the different Kefiran formulations and controls was performed using an injectability measurement device (KD Scientific, Portugal) commonly consisting of a syringe pump with a plastic syringe (1 mL) and a needle gauge of 21. The syringe was filled up with the different formulation solutions working in extrusion mode with a rate of 1 mL/min.

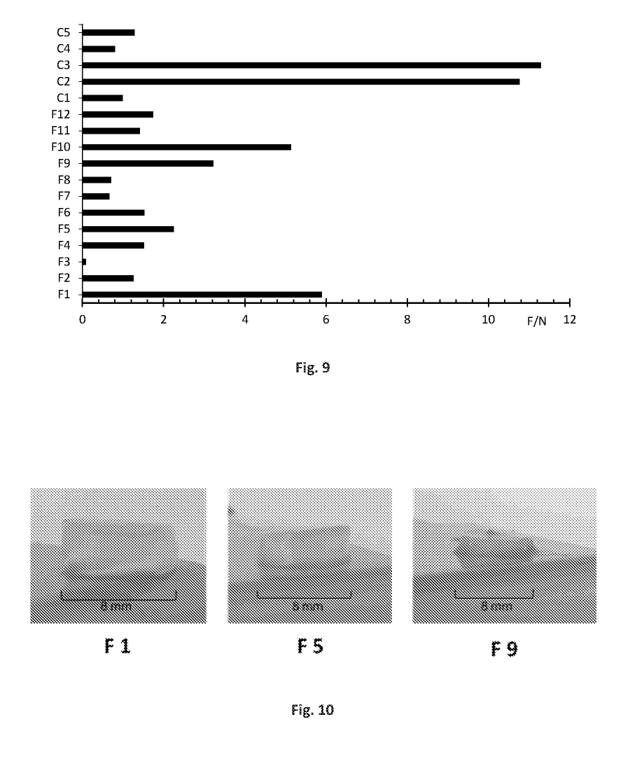

In an embodiment, the different Kefiran formulations and controls presented different values of force required for injection (FIG. 9). The extrusion force of hyaluronic acid (11.3 N) which presented the highest value, is clinically relevant data since the physician must inject the hyaluronic acid through a thin needle into soft tissue. FIG. 9 showed that significantly low force and homogeneous extrusion of Kefiran with or without other biomaterials versus HA.

In an embodiment, it has been shown through FIG. 9 that Kefiran formulations could represent interesting viscosupplementation products over HA intra-articular injections since they offer a smooth injectability by low extrusion forces.

In an embodiment, same Kefiran formulation cryogels formulations (as described on Table VI) were performed in order to produce Kefiran cryogels. Kefiran formulations and controls were prepared at concentration of 2% w/V in ultrapure H₂O. The solutions were transferred to silicone molds h 2 mm×d 8 mm; then the molds were placed immediately in a freezer at −20° C. for 24 hrs and transferred to a refrigerator at 4° C. for a further 24 hrs.

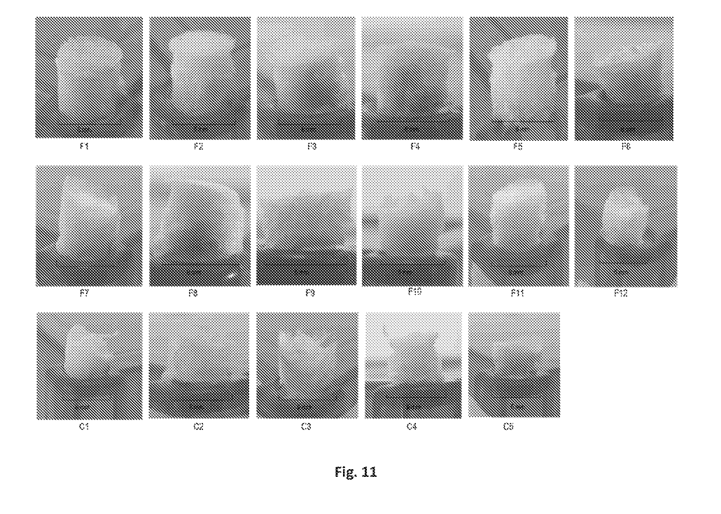

In an embodiment, the cryogels that have been observed with a remarkable structure were those from the formulations Kefiran+Gellan Gum. The best formulation seemed to be the one that has 75% of Kefiran and 25% of Gellan Gum (FIG. 10).

For the majority of cells, the natural environment is a tissue extracellular matrix, which is generally a type of hydrogel. Hydrogels of natural polymers, especially polysaccharides due to their properties make them very interesting for tissue engineering and regenerative medicine (Vashist and Ahmad 2015). Kefiran cryogels could act as a substitute for conventional tissue engineering materials having improved and restored tissue function.

In an embodiment, same Kefiran formulation scaffolds were performed. Properties of the biomaterial, such as its chemical composition, surface charge, hydrophobicity, surface roughness and the presence of specific proteins at the surface, are all considered to be essential in the initial cell attachment process (Ribeiro, Monteiro et al. 2012).

In an embodiment, same formulations were performed in order to produce Kefiran scaffolds (Table VI). Kefiran formulations and controls were prepared at concentration of 2% w/V in ultrapure H₂O. The solutions were transferred to an adapted 96-well microplate; then the microplates were placed immediately in a freezer at −20° C. for 24 hrs and transferred to a refrigerator at 4° C. for a further 24 hrs. Finally, the different scaffolds in the microplates were freeze-dried.

In an embodiment, same Kefiran formulation scaffolds were performed—All the scaffolds were obtained using a 96-well microplate. Designing of scaffolds with interesting and ideal characteristics, such those found in Kefiran polysaccharide, is the main key factor for successful tissue engineering. Recently, polysaccharide hydrogels have received a significant interest as leading candidates for engineered tissue scaffolds due to their unique structural and compositional similarities to the natural extracellular matrix, in addition to their appropriate structure for cellular survival and proliferation.

In an embodiment, in order to understand the mechanisms of action of Kefiran polysaccharides over different cells, two more different Kefiran extraction protocols detailed below (Method 2, K2—Kefiran obtain by method 2 and Method 3, K3—Kefiran obtain by method 3) were realized in addition to the previous one (Method 1, K1—Kefiran obtain by method 1); and several in vitro assays were performed.

In an embodiment, it is disclosed the isolation of Kefiran polysaccharides from supernatant (Method 2). Kefiran polysaccharides (K2—Kefiran obtained by method 2) were isolated from a different method previously described (Maalouf, Baydoun et al. 2011) with major modifications. Pasteurized skimmed milk (150 mL) was inoculated with kefir grains (50 g). Inoculated milk samples were incubated at room temperature for 24 hrs in a sealed-glass container. At the end of fermentation, the milk was strained to remove the kefir grains. The yeast and bacteria in the filtrate were removed by centrifugation (18,300 G for 20 min at 4° C.). One part of the supernatant was stored at −20° C. until needed for treatment of cells (K2L), the other one was freeze-dried (K2S).

In an embodiment, it is disclosed the isolation of Kefiran polysaccharides from supernatant (Method 3). Kefiran polysaccharides (K3—Kefiran obtained by method 3) were isolated from a different method previously described (Ye et al., 2009) with major modifications. The culture medium was separated by centrifugation (18,300 G, 20 min). The supernatant was collected and mixed with 3 volumes of cold ethanol absolute (v/V), and left overnight at 4° C. for polysaccharide isolation. The precipitate was rinsed thoroughly with water, filtered and then dried at 60° C. The crude EPS isolated was dissolved in distilled water and further treated with Sevage reagent (chloroform:n butanol at 5:1, v/V) for 2 times to remove the residual protein. The EPS, which was in supernatant, was purified again by ethanol absolute and left overnight at −20° C. The resulting precipitate was dissolved in distilled water and dialyzed.

In an embodiment, analysis of Kefiran's cytotoxicity using a cell line was performed. L929 cell line from mouse were used to evaluate Kefiran (K1, K2 and K3) cytotoxicity as described in the ISO 10993-5 (2009).

In an embodiment, in the first day of experiment, 10 000 cells were seeded in each well of a 96-well plate. Then, in accordance with ISO 10993-12 (2012). Kefiran samples were prepared: 4% w/V of Kefiran 1 (first extraction procedure, K1) in 0.9% w/V of NaCl; 4% w/V Kefiran 2 (second extraction procedure, liquid part, K2L) in 0.9% w/V of NaCl; and the one freeze-dried (second extraction procedure, K2S); and 4% w/V of Kefiran 3 (third extraction, K3) in 0.9% w/V of NaCl; and milk used for the extractions. In the next day, the culture medium was replaced for each of the previous solutions diluted in culture medium at final concentration of 1% v/V. The culture medium was composed of low-glucose Dulbecco's Modified Eagle Medium (DMEM, Sigma) supplemented with 10% v/V Fetal Bovine Serum (FBS, Invitrogen) and 1% v/V antibiotic/antimycotic (Invitrogen). Sample composed of culture medium was used as a negative control (Ctrl−). A positive control (Ctrl+) composed of Triton X-100 (Sigma-Aldrich) at a concentration of 1% v/V in culture medium was used. Cultures were maintained at 37° C. under a humidified atmosphere of 5% v/V CO₂ in air. Finally, at 24, 48 and 72 hrs of culture, cell growth, cell proliferation and cell damage were analysed in this experiment.

In an embodiment, cell growth was assessed using the CellTiter 96® AQueous One Solution Cell Proliferation Assay (MTS, Promega). At each time point, 24, 48 and 72 hrs cells were incubated with 20% v/V of MTS in culture medium without phenol red (Sigma) for 3 hrs at 37° C. The supernatant was then transferred to a new 96-well plate and absorbance measurements were carried out using a microplate reader (Biotek Synergy HT) at 490 nm.

In an embodiment, cell proliferation was assessed by total double-stranded DNA (dsDNA) quantification. At each time point, cells were incubated for 1 hr at 37° C. in ultrapure H₂O. Then, cell lysates were transfer to a 1.5 mL tube and storage at −80° C. until analysed. Quant-iT PicoGreen dsDNA kit (Molecular Probes, Invitrogen) was used according to manufacturer's instructions. Briefly, samples were transferred to a 96-well white plate and diluted in TE buffer. After adding the Quant-iT PicoGreen dsDNA reagent, samples were incubated for 10 min at RT in the dark, and fluorescence was quantified using a microplate reader (Biotek Synergy HT) with Ex/Em at 480/530 nm. R.F.U. were converted into ng/mL using a standard curve of DNA in the range of 1-2,000 ng/mL.

In an embodiment, cell damage was studied through F-actin staining. For that, cells were washed with phosphate buffer saline (PBS, Sigma-Aldrich), fixed with 10% Neutral Buffered Formalin (ThermoFisher Scientific) for 15 min and permeabilized for 5 min with 0.1% v/V Triton X-100 (Sigma-Aldrich) in PBS. Afterwards, samples were incubated for 30 min in 1% w/V BSA (Sigma-Aldrich) in PBS to block unspecific binding. F-actin filaments were stained with Phalloidin-Tetramethylrhodamine B isothiocyanate (Sigma-Aldrich, 1:40) and nuclei were counterstained with 1:5000 of the stock of 4,6-Diamidino-2-phenyindole, dilactate solution (DAPI, 1 mg/mL, Biotium). Samples were analysed by fluorescence inverted microscope (Zeiss Axio observer).

In an embodiment, to evaluate the cytotoxicity of Kefiran isolated from the tree different procedures (K1, K2 and K3) and is safety as an upcoming viscosupplementation material, ISO 10993-5 (2009) was followed. The purpose of this guidelines was to provide a test sample where the biological reactivity of any leachable could be detected.

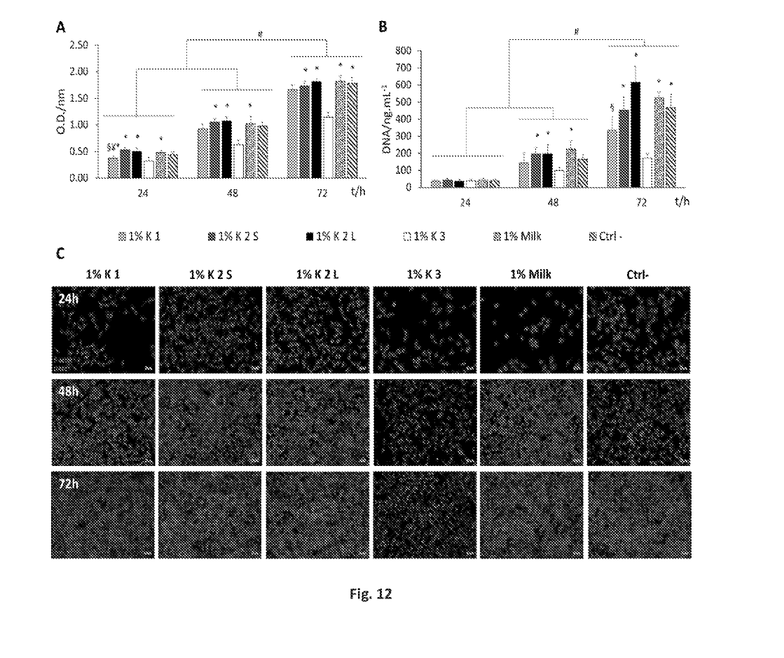

In an embodiment, cell growth, cell proliferation and cell damage were evaluated as depicted in FIG. 12. FIG. 12 A showed cell growth assessed by MTS assay, once the absorbance was directly proportional to the number of living cells within the culture.

In an embodiment, after 24 hrs of culture the K1 showed lower number of living cells compared with K2L, K2S and K3. But this difference was not both observed at 48 hrs and 72 hrs of culturing. Importantly, K3 presented the lowest number of living cells of all, showing differences from K2, milk and Ctrl−. It is important to mention that along the culture, the number of living cells increased, as corroborated by dsDNA quantification and F-actin staining (FIGS. 12 B and 12 C). Moreover, both assays showed that cells submitted to Kefiran 3 proliferated at lower rates than cells submitted to Kefiran 1 and Kefiran 2. Finally, F-actin staining showed no damage on cells when submitted to any condition (FIG. 12 C).

In an embodiment, human adipose derived stem cells (hASCs) were obtained from human adipose tissue after liposuction procedure, which were performed at Hospital da Prelada (Porto, Portugal), after patient's informed consent and under a collaboration protocol approved by the ethical committees of both institutions. In order to isolate the hASCs, the adipose tissue was submitted to the action of 0.05% collagenase type II (Sigma), under agitation for 1 hr at 37° C. Then, it was filter with a strainer and centrifuged at 800 G for 10 min. After discarded the supernatant, pellets were resuspended in PBS and centrifuged at 350 G for 5 min. Finally, the cell pellet was resuspended in Minimum Essential Media α (α-MEM, Gibco), supplemented with 10% fetal bovine serum (FBS, Invitrogen), and 1% antibiotic/antimycotic (Invitrogen). Cultures were maintained at 37° C. under a humidified atmosphere of 5% v/V CO₂ in air. hASCs were selected by plastic adherence and passage at 80% confluence. In the different studies, hASCs in passage 4 were used for this assay.

In an embodiment, the evaluation of cytotoxicity of Kefiran polysaccharides (K1, K2S and K2L) using hASCs was performed as described for L929 cells line, following ISO 10993-12 (2012) guidelines.

In an embodiment, hASCs were seeded in each well of a 96-well plate at a density of 3000 cells/cm². Then, in accordance with ISO 10993-12 (2012), samples were prepared: 4% w/V K1 in 0.9% w/V of NaCl from first extraction procedure, hereafter designated Kefiran 1; 4% w/V K2L and K2S in 0.9% w/V of NaCl from second extraction procedure. In the next day, the culture medium was replaced for each of the previous solutions diluted in culture medium at final concentration of 1% v/V. Additionally, a negative control (Ctrl−) was prepared composed of culture medium and a positive control (Ctrl+) composed of Triton X-100 (Sigma-Aldrich) at a concentration of 1% v/V in culture medium. Cultures were maintained at 37° C. under a humidified atmosphere of 5% v/V CO₂ in air. Finally, at 24, 48 and 72 hrs of culture, cell growth and cell damage were analysed as described above.

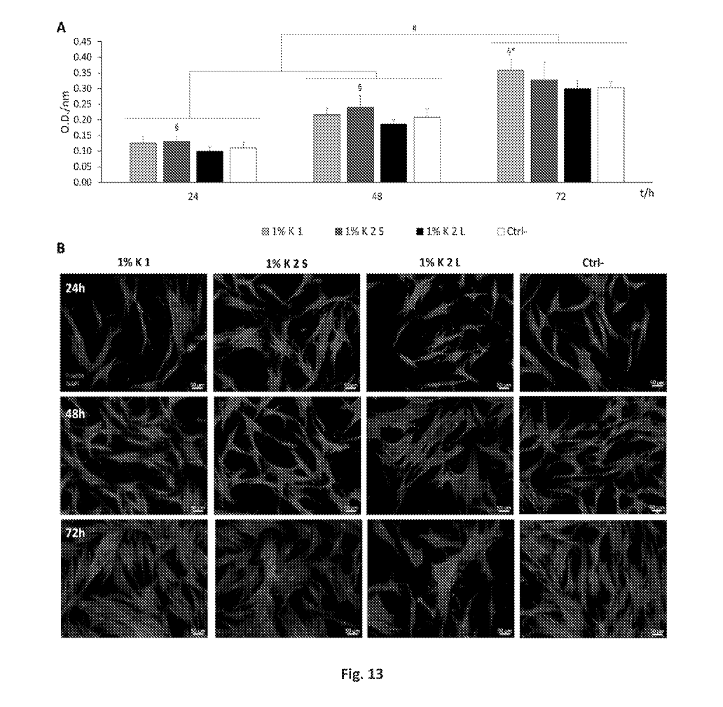

In an embodiment, after the evaluation of cytotoxicity using a cell line, the cytotoxicity was analysed using primary cells. In fact, foreseeing the use of Kefiran as a viscosupplement, the cytotoxicity was analysed in a more physiologically relevant environment, i.e. using human primary cells. Nevertheless, in this assay only K1, K2L and K2S were analysed. In FIG. 13, it was possible to observe cell growth, assessed by MTS assay, cell proliferation, assessed by dsDNA quantification, and cell damage, assessed by F-actin staining, along 72 hrs of culture. The number of living cells increased along the time of culture, but cells cultured with K2L showed less living cells than Kefiran 1 and Kefiran 2 as depicted in FIG. 13 A. These observations were corroborated by F-actin staining as illustrated in FIG. 13 B. Furthermore, after 72 hrs of culture it was observed more living cells in Kefiran 1 than Kefiran 2. Interestingly, there was a tendency to Kefiran 1 and Kefiran 2 conditions which presented higher number of living cells than Ctrl−.

In an embodiment, analyze of biocompatibility of Kefiran scaffolds was performed. Biocompatibility of Kefiran (K1 from extraction Method 1) scaffolds produced using different formulations (Table VI) was analysed using hASCs. For that, hASCs were seeded on top of each scaffold at a density of 100,000 cells/cm². The same number of cells cultured in a 24-well plate were use as control (Ctrl). Cultures were maintained at 37° C. under a humidified atmosphere of 5% v/V CO₂ in air. At 24 hrs and 72 hrs of culturing, cell's metabolic activity was assessed.

In an embodiment, cell metabolic activity was assessed using AlamarBlue® assay (Bio Rad). At each time point, 24 and 72 hrs, scaffolds were incubated with 20% v/V of AlamarBlue® reagent in medium for 4 hrs at 37° C. The supernatant was then transferred to a 96-well black plate and fluorescence measurements were carried out using a microplate reader (Biotek Synergy HT) with Ex/Em at 530/590 nm. For normalization of AlamarBlue® results, total double-stranded DNA (dsDNA) was quantify. First, scaffolds were recovered at each time point, incubated for 1 hr at 37° C. in ultrapure water and storage at −80° C. until analysed. Scaffolds in ultrapure water were then sonicated during 15 min and used for dsDNA quantification using the Quant-iT PicoGreen dsDNA kit (Molecular Probes, Invitrogen), according to manufacturer's instructions. Briefly, samples were transferred to a 96-well white plate and diluted in TE buffer. After adding the Quant-iT PicoGreen dsDNA reagent, samples were incubated for 10 min at RT in the dark, and fluorescence was quantified using a microplate reader with Ex/Em at 480/530 nm. RFUs were converted into ng/mL using a standard curve of DNA in the range of 1-2,000 ng/mL. Finally, these results were used to normalize AlamarBlue® results.

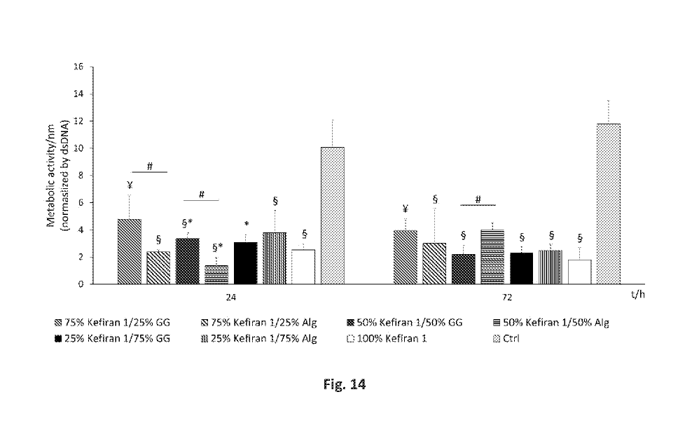

After the evaluation of cytotoxicity of Kefiran extracted using different protocols, scaffolds were prepared. For that, Kefiran 1 was chosen and mixed with other natural polymers, gellan gum (GG) and alginate (Alg) at different concentrations and their biocompatibilities were studied. For that, metabolic activity of cells cultured on top of scaffolds was analysed and normalized by dsDNA content, as depicted in FIG. 14.

After 24 hrs, cell cultured on top of scaffolds of 75% Kefiran 1/25% (w/V) GG or 50% (w/V) Kefiran 1/50% (w/V) GG showed higher metabolic activity than 75% Kefiran 1/25% Alg or 50% Kefiran 1/50% Alg, but this difference was not observed after 72 hrs of culture. In fact, at this time point 50% (w/V) Kefiran 1/50% (w/V) Alg showed higher metabolic activity than 50% Kefiran 1/50% (w/V) GG. Noteworthy, only cells cultured on 75% (w/V) Kefiran 1/25% (w/V) GG scaffolds showed higher metabolic activity than 100% (w/V) Kefiran 1 scaffolds at 24 hrs and 72 hrs of culture. Additionally, Ctrl demonstrated higher metabolic activity than cells culture on these scaffolds as expected and as described for cells cultured on scaffolds with high water content.

Due to its aforementioned remarkable properties, Kefiran appears thus as a relevant candidate for the preparation of such biomaterials. Kefiran biopolymer allows cell attachment and proliferation, demonstrating that it could substitute for missing or damaged tissue.

In an embodiment, statistical analyses were performed using GraphPad Prism 6.0 software. The non-parametric Mann-Whitney test was used to compare two groups, whereas comparison between more than two groups was performed using the Kruskal-Wallis test followed by Dunn's comparison test. The critical level of statistical significance chosen was p<0.05.

All references recited in this document are incorporated herein in their entirety by reference, as if each and every reference had been incorporated by reference individually.

Those skilled in the art will recognize, or be able to ascertain using no more than routine experimentation, many equivalents to the specific embodiments of the invention described herein. The scope of the present invention is not intended to be limited to the above description, but rather is as set forth in the appended claims.

Where singular forms of elements or features are used in the specification of the claims, the plural form is also included, and vice versa, if not specifically excluded. For example, the term “a polysaccharide” or “the polysaccharide” also includes the plural forms “polysaccharides” or “the polysaccharides”, and vice versa. In the claims articles such as “a”, “an”, and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention also includes embodiments in which more than one, or all of the group members are present in, employed in, or otherwise relevant to a given product or process.

Furthermore, it is to be understood that the invention encompasses all variations, combinations, and permutations in which one or more limitations, elements, clauses, descriptive terms, etc., from one or more of the claims or from relevant portions of the description is introduced into another claim. For example, any claim that is dependent on another claim can be modified to include one or more limitations found in any other claim that is dependent on the same base claim.

Furthermore, where the claims recite a composition, it is to be understood that methods of using the composition for any of the purposes disclosed herein are included, and methods of making the composition according to any of the methods of making disclosed herein or other methods known in the art are included, unless otherwise indicated or unless it would be evident to one of ordinary skill in the art that a contradiction or inconsistency would arise.

Where ranges are given, endpoints are included. Furthermore, it is to be understood that unless otherwise indicated or otherwise evident from the context and/or the understanding of one of ordinary skill in the art, values that are expressed as ranges can assume any specific value within the stated ranges in different embodiments of the invention, to the tenth of the unit of the lower limit of the range, unless the context clearly dictates otherwise. It is also to be understood that unless otherwise indicated or otherwise evident from the context and/or the understanding of one of ordinary skill in the art, values expressed as ranges can assume any subrange within the given range, wherein the endpoints of the subrange are expressed to the same degree of accuracy as the tenth of the unit of the lower limit of the range.

In addition, it is to be understood that any particular embodiment of the present invention may be explicitly excluded from any one or more of the claims. Where ranges are given, any value within the range may explicitly be excluded from any one or more of the claims. Any embodiment, element, feature, application, or aspect of the compositions and/or methods of the invention, can be excluded from any one or more claims. For purposes of brevity, all of the embodiments in which one or more elements, features, purposes, or aspects is excluded are not set forth explicitly herein.

The above described embodiments are combinable.

The following claims further set out particular embodiments of the disclosure.

The following references are herewith incorporated in their entirety.
Botelho, P. S, M. I. Maciel, L. A. Bueno, Mde F. Marques, D. N. Marques, T. M. Sarmento Silva (2014). Characterisation of a new exopolysaccharide obtained from of fermented kefir grains in soymilk. Carbohydrate Polymers, 107:1-6.
Ghasemlou, M., F. Khodaiyan, A. Oromiehie, M. S. Yarmand (2011). Characterization of edible emulsified films with low affinity to water based on Kefiran and oleic acid. International Journal of Biological Macromolecules, 49 (3):378-384.
Piermaria, J. A., A. Pinotti, M. A. Garcia, A. G. Abraham (2009). Films based on Kefiran, an exopolysaccharide obtained from kefir grain: Development and characterization. Food Hydrocolloid, 23 (3):684-690.
Pop, O. L., T. Brandau, J. Schwinn, D. C. Vodnar, C. Socaciu (2015). The influence of different polymers on viability of Bifidobacterium lactis 300b during encapsulation, freeze-drying and storage. Journal of Food Science and Technology, 52 (7):4146-4155.
Zajsek, K. and A. Gorsek (2011). Experimental Assessment of the Impact of Cultivation Conditions on Keflran Production by the Mixed Microflora Imbedded in Kefir Grains. Chemical Engineering Transactions, 24:481-486.
Amin, A. R. and A. B. Islam (2014). Genomic analysis and differential expression of HMG and S100A family in human arthritis: upregulated expression of chemokines, IL-8 and nitric oxide by HMGB1, DNA and Cell Biology, 33 (8): 550-565.
Aparadh, V. T., V. V. Naik and B. A. Karadge (2012). Antioxidantive properties (TPC, DPPH, FRAP, Metal chelating ability, reducing power and TAC) within some cleome species. Annali di botanica 2: 49-56.
Cilurzo, F., F. Selmin, P. Minghetti, M. Adami, E. Bertoni, S. Lauria and L. Montanari (2011). Injectability evaluation: an open issue. AAPS PharmSciTech, 12 (2): 604-609.
El, S. N. and S. Karakaya (2004). Radical scavenging and iron-chelating activities of some greens used as traditional dishes in Mediterranean diet. International journal of food sciences and nutrition, 55 (1): 67-74.
Flora, S. J. (2009). Structural, chemical and biological aspects of antioxidants for strategies against metal and metalloid exposure. Oxidative Medicine and Cellular Longevity, 2 (4): 191-206.
Gutierrez, R. M. P. and E. G. Baez (2014). Evaluation of antidiabetic, antioxidant and antiglycating activities of the Eysenhardtia polystachya. Pharmacognosy magazine, 10 (Suppl 2): 5404.
Liu, S. H., S. W. Sun, Z. F. Tian, J. Y. Wu, X. L. Li and C. P. Xu (2015). Antioxidant and Hypoglycemic Activities of Exopolysaccharide by Submerged Culture of Inocutus Hispidus. Indian journal of pharmaceutical sciences, 77 (3): 361-365.
Maalouf, K., E. Baydoun and S. Rizk (2011). Kefir induces cell-cycle arrest and apoptosis in HTLV-1-negative malignant T-lymphocytes. Cancer Management and Research, 3: 39-47.
Nagai, T., T. Nagashima, N. Suzuki and R. Inoue (2005). Antioxidant activity and angiotensin I-converting enzyme inhibition by enzymatic hydrolysates from bee bread. Zeitschrift fur Naturforschung. Journal of biosciences, 60 (1-2): 133-138.
Piermaria, J. A., A. Pinotti, M. A. Garcia and A. G. Abraham (2009). Films based on Kefiran, an exopolysaccharide obtained from kefir grain: Development and characterization. Food Hydrocolloids, 23 (3): 684-690.
Qi, H., Q. Zhang, T. Zhao, R. Hu, K. Zhang and Z. Li (2006). In vitro antioxidant activity of acetylated and benzoylated derivatives of polysaccharide extracted from Ulva pertusa (Chlorophyta). Bioorganic & medicinal chemistry letters, 16: 2441-2445.
Ribeiro, M., F. J. Monteiro and M. P. Ferraz (2012). Infection of orthopedic implants with emphasis on bacterial adhesion process and techniques used in studying bacterial-material interactions. Biomatter, 2 (4): 176-194.
Saturnino, C., M. S. Sinicropi, O. I. Parisi, D. lacopetta, A. Popolo, S. Marzocco, G. Autore, A. Caruso, A. R. Cappello, P. Longo and F. Puoci (2014). Acetylated hyaluronic acid: enhanced bioavailability and biological studies. BioMed Research International, 2014: 921549.
Singhal, M. and P. Ratra (2013). “Antioxidant Activity, Total Flavonoid and Total Phenolic Content of Musa acuminate Peel Extracts.” Global Journal of Pharmacology 7 (2): 118-122.
Telles, C. B. S., D. A. Sabry, J. Almeida-Lima, M. S. S. M. Costa, R. F. Melo-Silveira, E. S. Trindade, G. L. Sassaki, E. Wisbeck, S. A. Furlan, E. L. Leite and H. A. O. Rocha (2011). Sulfation of the extracellular polysaccharide produced by the edible mushroom Pleurotus sajor-caju alters its antioxidant, anticoagulant and antiproliferative properties in vitro. Carbohydrate Polymers, 85 (3): 514-521.
Vashist, A. and S. Ahmad (2015). Hydrogels in tissue engineering: scope and applications. Current Pharmaceutical Biotechnology, 16 (7): 606-620.
Wang, Y., Z. Yang and X. Wei (2012). Antioxidant activities potential of tea polysaccharide fractions obtained by ultra filtration. International journal of biological macromolecules, 50 (3): 558-564.
Ye, S. H., F. Liu, J. H. Wang, H. Wang, M. P. Zhang (2012). Antioxidant activities of an exopolysaccharide isolated and purified from marine pseudomonas pf-6. Carbohydrate Polymers. 87 (1):764-770.

1. A Kefiran for use in regenerative medicine and/or tissue engineering wherein the Kefiran molecular weight is between 200-2,000 K Da.

2. The Kefiran according to claim 1, wherein the Kefiran molecular weight is between 1,000-1,720 kDa.

3. A method for treating or preventing a bone, cartilage, cornea, skin, vascular tissue, peripheral nerve, spinal cord or brain disease in a patient comprising administering the Kefiran of claim 1 to the patient.

4. The method of claim 3, wherein the disease is a bone or cartilage disease or defect.

5. A pharmaceutical composition comprising the Kefiran according to claim 1 in a therapeutically effect amount and a pharmaceutically acceptable excipient for use in the treatment of diseases that involve the regeneration of tissues or the treatment of tissues.

6-7. (canceled)

8. The pharmaceutical composition according to claim 5 comprising 0.1-50% (w/V) of Kefiran.

9. The pharmaceutical composition according to claim 8 comprising 1-5% (w/V) of Kefiran.

10. The pharmaceutical composition according to claim 8 comprising a further polysaccharide, a hydrogel, a protein, a therapeutic agent or combinations thereof.

11. The pharmaceutical composition according to claim 10 wherein said the polysaccharide or hydrogel is selected from the group consisting of: cellulose; alginate; chitosan, chondroitin sulphate, hyaluronic acid, gellan gum, dextran, collagen, guar gum, carrageenan, heparin, and mixtures thereof.

12. (canceled)

13. The pharmaceutical composition according to claim 10 wherein said therapeutic agent is selected from the group consisting of: antioxidant, anti-inflammatory, antipyretic, analgesic, anticancer, growth-factor, and mixtures thereof.

14. The pharmaceutical composition according to claim 5 wherein the composition further comprises a cell culture media, cell or mixtures thereof; in particular stem cell.

15. The pharmaceutical composition according to claim 5 wherein the composition is an injectable form.

16. The pharmaceutical composition according to claim 10 further comprising a plurality of hydrogels.

17. The pharmaceutical composition according to claim 5 further comprising an antiinflammatory agent, an antiseptic agent, an antipyretic agent, an anaesthetic agent, a therapeutic agent, or mixtures thereof.

18. A scaffold for the use in regenerative medicine or tissue comprising the Kefiran of claim 1.

19. A viscosupplement for the use in regenerative medicine or tissue comprising the Kefiran of claim 1.

20. A method of treating a patient suffering from a musculoskeletal disease, the method comprising administering an effective amount of the Kefiran of claim 1 to the patient.

21. The method of claim 20, wherein the administering is by intra-articular injection into a joint of the patient.

22. The pharmaceutic composition of claim 13, wherein the anti-inflammatory is diclofenac [2-(2,6-dichloroamino)phenyl]acetic acid.
